(19) United States
(12) Patent Application Publication
(10) Pub. No.: US 2019/0167536 A1
(43) Pub. Date: Jun. 6, 2019

(54) SOFT SOLID FIXED FORM COSMETIC MASK, INCLUDING COSMETIC OTC MASK, DELIVERY COMPOSITION AND APPARATUS

(71) Applicant: Yes To, Inc., Pasadena, CA (US)

(72) Inventor: Ingrid Karine Jackel, Pasadena, CA (US)

(21) Appl. No.: 15/830,431

(22) Filed: Dec. 4, 2017

(51) Int. Cl. A61K 8/02; A61Q 19/00; A61K 8/31; A61K 8/368; A61K 8/9789; A61K 8/67; A45D 40/04

(52) U.S. Cl. CPC A61K 8/0229; A61Q 19/00; A61K 8/31; A61K 8/368; A61K 2800/28; A61K 8/676; A45D 40/04; A61K 2800/87; A61K 8/9789

(57) ABSTRACT

Fixed, soft solid cosmetic mask products, including cosmetic OTC mask products, and delivery forms are disclosed. The mask formulation is capable of maintaining its shape until applied, and being put off during application in a relatively uniform layer.

### FIELD

Disclosed herein are soft solid fixed form mask compositions and delivery forms. In particular, cosmetic masks, including cosmetic OTC masks, formulated for stick delivery forms and those stick forms are disclosed herein.

### BACKGROUND

Cosmetic masks, including cosmetic OTC masks, such as facial masks or body masks, of various composition and for various purposes are well-known in the art. Some provide anti-aging effect, anti-acne treatment, pore cleaning, or other purported effects. Some are hydrogel based, some “mud”-based, emulsion based, paper/non-woven based, powder based, gel based, clay based, etc. Some come in one, two, or more part compositions, some in the form of a “mud,” some as an emulsion, paste, gel, clay, powder, liquid, etc. applied topically to the skin. The mask formula is applied on the surface of the skin and, depending on the type of formula, can dry on the skin or can be rubbed on the skin and/or absorbed. Depending on the type of mask, the mask is left in place for various amounts of time to achieve the desired results. Depending on the formula, the mask can be removed by peeling, rinsing, or other techniques.

The application techniques used for this variety of masks has traditionally been the same, application via the hand or perhaps a tool, such as a cosmetic or mask spatula or brush, to the skin. The compositions, as noted above, typically range from a paste, clay, powder, gel, paper/non-woven, liquid, etc. Due to their consistencies, these mask formulas are often contained in jars, bottles, sachets, squeeze tubes, or the like, since their form ranges from liquid to paste. As can be appreciated by anyone who has used a mask, application can be messy or imprecise, leading to a less than optimal experience. Thus, it would be advantageous to have a mask formula in a fixed form, such as a soft solid, that easily glides onto the skin, hands-free, in a relatively uniform layer, thus minimizing any associated mess on the hands or surrounding environment, and improving ease of application.

Disclosed herein are such soft solid fixed form mask compositions and mask stick delivery forms.

### SUMMARY

Some embodiments provide a cosmetic mask stick product, including a cosmetic OTC mask stick product, comprising a soft solid fixed form cosmetic mask formulation capable of maintaining a solid shape until applied to the skin; whereupon the composition is paid out in a substantially uniform layer; a dispenser housing defining an open end and containing the soft solid fixed form cosmetic mask formulation, including cosmetic OTC mask formulation, for delivery through the open end.

In some embodiments, the dispenser housing is provided with a dispensing mechanism for selectively advancing the soft solid fixed form cosmetic mask formulation within and out of the dispenser housing.

In some embodiments, the dispensing mechanism is a screw-thread mechanism.

In some embodiments, the soft solid fixed form cosmetic mask formulation, including cosmetic OTC mask formulation, comprises a skin care and aesthetic portion; and a structural and functional portion.

In some embodiments, the structural and function portion comprises one or more filler, binder, wax, oil, solvent, solubilizer, humectant, hydrogel, surfactant, thickening agent, thinning agent, melting agent, activator, polymer, or other ingredients useful for adding, developing, and maintaining a soft solid fixed form, and any combination thereof.

In some embodiments, the cosmetic and aesthetic portion comprises one or more of charcoal, salicylic acid, tomato extract, or a combination thereof.

In some embodiments, the cosmetic and aesthetic portion comprises a charcoal, salicylic acid, and a tomato extract.

In some embodiments, the cosmetic and aesthetic portion comprises one or more of grapefruit-based ingredient, vitamin C, or a combination thereof.

In some embodiments, wherein the cosmetic and aesthetic portion comprises a grapefruit-based ingredient and vitamin C.

Some embodiments further comprise one or more exfoliant.

In some embodiments, the skin care and aesthetic portion further comprises one or more moisturizer, exfoliant, purifying agent, charcoal, activated carbon, fruit extract or essence, vegetable extract or essence, essential oil, anti-aging ingredient, enzyme, vitamin, mineral, whitening agent, bronzing agent, spot remover, anti-wrinkling agent, humectant, hyaluronic acid, anti-acne ingredients, etc., or combination thereof.

In some embodiments, the cosmetic mask, including cosmetic OTC mask, stick formulation comprises at least about 95% by weight natural ingredients by weight of the formulation, excluding water.

Some embodiments provide a cosmetic stick comprising a cosmetic mask formulation, including a cosmetic OTC mask formulation, in a soft solid fixed form.

Some embodiments provide a cosmetic mask composition, including a cosmetic OTC mask composition, in a mask stick form having the properties of a soft solid fixed form capable of maintaining its solid shape which allows for direct application of the mask composition to the skin; whereupon the composition is paid out in a substantially uniform layer.

Some embodiments include such a composition in a container or housing for ease of storage and application and travel.

Some embodiments provide a cosmetic mask stick formulation, including a cosmetic OTC mask stick formulation, comprising a skin care and aesthetic portion; and a structural and functional portion; in the form of a soft solid fixed form capable of maintaining its solid shape until applied to the skin; whereupon the composition is paid out in a substantially uniform layer.

These and other variations will be apparent to those of ordinary skill in the art upon reading this disclosure.

### BRIEF DESCRIPTION OF THE DRAWINGS

FIG. 1 is a perspective view of an embodiment of a mask stick product in a screw dispenser.

FIG. 2 is a front view of an exemplary mask stick product in a screw dispenser.

FIG. 3 is a top view of a mask stick product in a screw dispenser, according to one embodiment.

FIG. 4 is a bottom view of an exemplary mask stick product in a screw dispenser.

FIG. 5 is a cross-sectional front view of the exemplary mask stick product in a screw dispenser with a screw-thread advancement mechanism without the outer cap and inner cup.

FIG. 6 is a cross-sectional side view of a mask stick product in a screw dispenser with a screw-thread advancement mechanism, according to an embodiment.

FIG. 7 is a cross-sectional view of an exemplary cap for a screw dispenser.

FIG. 8 is a cross-sectional view of an exemplary inner cap for a screw dispenser.

FIG. 9 is a photograph of an exemplary prototype mask stick product with a screw dispenser.

### DETAILED DESCRIPTION

Although cosmetic masks, including cosmetic OTC masks, and soft solid fixed form delivery systems, i.e. a stick, are each separately known, the concept of a cosmetic mask, including a cosmetic OTC mask, composition in a soft solid fixed form, i.e. a mask stick, is new. Heretofore, no one has attempted to combine these two into a soft solid fixed form cosmetic mask formulation, including cosmetic OTC mask formulation, or mask stick delivery form. It is the identification of the need and possibility of such mask sticks that forms part of the inventive concept. Only upon that recognition can one decide how to achieve such a product in a mask stick form. Applicant has identified the need and benefits of such a mask stick form, and has pursued formulations to achieve the desired result, from readily available materials and techniques. This disclosure relates to a mask stick comprising a cosmetic mask composition, including cosmetic OTC mask composition, in a soft solid fixed form, that is, a mask stick.

The mask stick need not be limited to a particular type of mask and may be used to facilitate application and handling of facial and body masks, hair masks, clay masks, mud masks, peel off masks, leave-on masks, rinse-off masks, exfoliating masks, rub-off masks, gel masks, and other types of masks. A unique aspect disclosed herein is that a mask formulation is placed in a stick component and is applied in a new, unique method for cosmetic masks, including cosmetic OTC masks.

This disclosure is written in the context of a skin or body mask and refers to application to the skin. Certain embodiments can include hair masks which are applied to the hair rather than to the skin. Those of skill in the art will recognize that reference to the skin is a reference to the surface to which the mask is applied, and in the case of a hair mask, that surface is the hair.

The soft solid fixed form mask stick described herein was developed to achieve the desired mask stick form having the desired stability, application, payoff, and other properties. The functional and structural ingredients were selected and modified to achieve the desired properties.

The soft solid fixed form mask composition and mask stick delivery forms as contemplated herein strikes a balance between being firm enough to hold their shape yet being capable of a substantially uniform product layer of the desired thickness and consistency suitable for the desired application. The application of the mask composition may, but not necessarily, be accomplished in a single application, e.g., swipe, stroke, etc. The soft solid fixed form mask composition is formulated as a stick form. It will be appreciated that the soft solid fixed form mask compositions disclosed herein, have unique properties and considerations for storing and applying a mask composition in a stick form. Those of skill in the cosmetics arts will recognize that by stick form it is meant a soft solid fixed form having a generally three-dimensional shape, such as a rectangular prism, a round or oval column, or other three dimensional, usual oblong shape.

Some unique properties or characteristics of the mask sick include intended length of application, since masks are generally applied and allowed to sit before being removed. Also, a cosmetic mask, including a cosmetic OTC mask, particularly a facial mask, must be applied with precision to avoid contact with the eyes or other sensitive areas. Cosmetic masks, including cosmetic OTC masks, also benefit from uniform application, since even drying or absorption is often important to the function of the mask and even application of the ingredients is beneficial.

A cosmetic mask stick composition, including a cosmetic OTC mask stick composition, generally comprises a list of ingredients that provide skin care, aesthetic, structural, and/or functional benefits.

The skin care and aesthetic portions may include any cosmetic ingredient, fragrance, skin treatment ingredient, cosmetic colorant, or any other ingredient useful in the cosmetic arts or in skin-treatment. A list of suitable ingredients that may be included in the skin care and aesthetic portion of the mask stick include: moisturizers, exfoliants, purifying agents, charcoal, activated carbon, fruit extracts or essences, vegetable extracts or essences, essential oils, anti-aging ingredients, enzymes, vitamins, minerals, whitening agents, bronzing agents, spot removers, anti-wrinkling agents, humectants, hyaluronic acid, anti-acne ingredients, etc. or combinations of such ingredients. In some embodiments, the ingredients can be substantially natural ingredients, or derived from natural sources.

The structural and functional portion includes those ingredients that determine the properties of the soft solid fixed form cosmetic mask, including a cosmetic OTC mask. This portion may include any number of ingredients chosen such that together, and when combined with the skin care and aesthetic portion, they provide a soft solid fixed form cosmetic mask delivery form, including cosmetic OTC mask delivery form, capable of paying out a relatively uniform layer of the formulation when swiped, stoked, etc., applied to the skin. The structural and functional ingredients may include: fillers, binders, waxes, oils, solvents, solubilizers, stabilizers, humectants, hydrogels, surfactants, thickening agents, thinning agents, melting agents, activators, polymers, or other ingredients useful for adding, developing, and maintaining a soft solid fixed form.

The cosmetic mask formulation, including cosmetic OTC mask formulation, can be made by mixing together the appropriate ingredients to achieve the desired cosmetic or therapeutic effect and to achieve the soft solid fixed form capable of paying out a relatively uniform layer of product when applied to the skin.

The soft solid fixed form cosmetic mask formulation, including cosmetic OTC mask formulation, is most effectively delivered in a stick form. In such embodiments, a housing or container, typically, but not necessarily made of plastic and provided with a mechanism for advancing product housed therein, will be used to hold and apply the cosmetic mask composition, including cosmetic OTC mask composition. Those of ordinary skill in the art will readily recognize suitable techniques for filling such containers. For example, the soft solid fixed form cosmetic mask formulation, or cosmetic OTC mask formulation, could be hot poured into the container, which acts as a mold and allowed to cool. Under such circumstances, the composition would cool into the soft solid fixed form cosmetic mask, including cosmetic OTC mask (i.e., mask stick), which would be solid in the container, and smoothly applied to the skin. The mask is allowed to sit a desired amount of time on the skin before removal by various techniques depending on the formulation.

In today's environment, consumers are ever more environmentally conscious and concerned about the use of chemicals, particularly on their skin. To this end, some embodiments herein are directed to natural formulas. By “natural formula,” it is meant that, excluding water, at least about 95% by weight of the formulation will be natural ingredients. When calculating the percentage of natural ingredients, the number should always be rounded up to the nearest whole number (e.g., 98.6 will be declared as 99%).

“Natural ingredients” include ingredients that come from or are made from a renewable resource found in nature (flora, fauna, mineral). Preferably such materials can be verified as such by their suppliers.

Importantly, some embodiments will be compliant with the Whole Foods Body Standard, meet EU natural personal care requirements EU Regulation (EC) 1223/2009, will be on the Australia Inventory of Chemical Substances (AICS), and will meet Canada's personal care requirements.

In some embodiments, the mask stick formulation will have antimicrobial effects. In such embodiments, the antimicrobial effect can be tested and confirmed via United States Pharmacopeial Convention Chapter 51: Antimicrobial Effectiveness Testing.

All embodiments should be suitable for contact, and in some embodiments prolonged contact, with the skin, particularly the skin of the face. One method to assess this is via the Shelanski Method also known as the Repeated Insult Patch Test (RIPT). Successful formulations will demonstrate little or no potential for eliciting dermal irritation or inducing sensitization under test conditions.

As noted above, the mask stick formulation should be stable for a specified period of time and maintain its soft solid fixed form until application. Stability testing and evaluation can be performed via known techniques.

In particular embodiments, if desired, the cosmetic and aesthetic portion can be adapted from an existing line of cosmetic products such as, but not limited to, Yes To's grapefruit line of products, or their Tomato Charcoal line of products, or other product lines. Of course, any suitable mask formulation could be adapted in accordance with this disclosure.

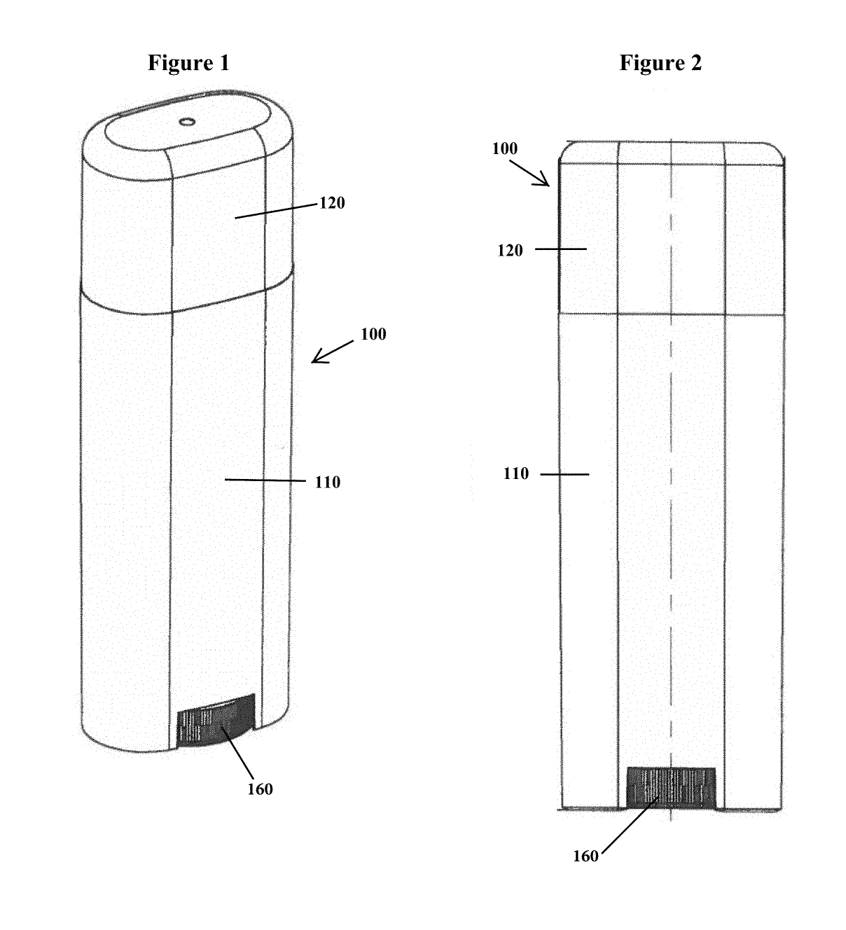

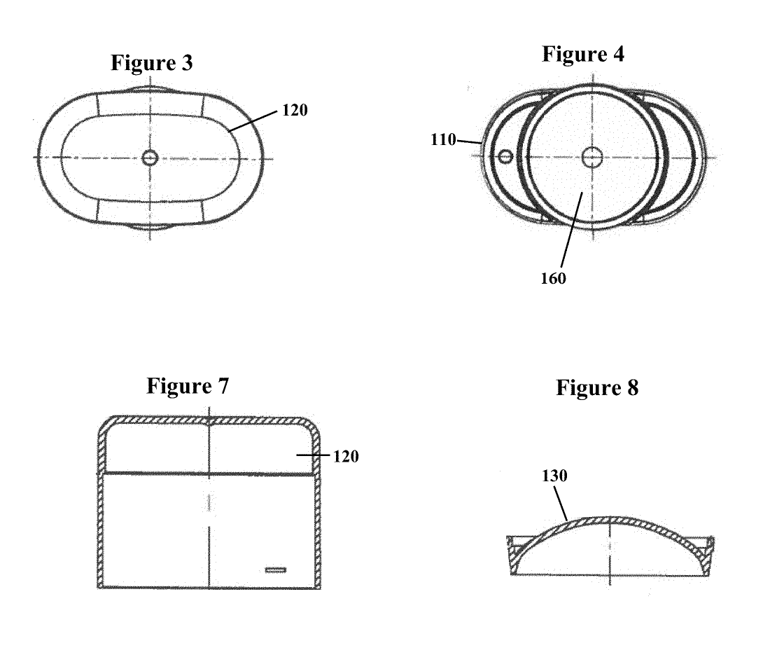

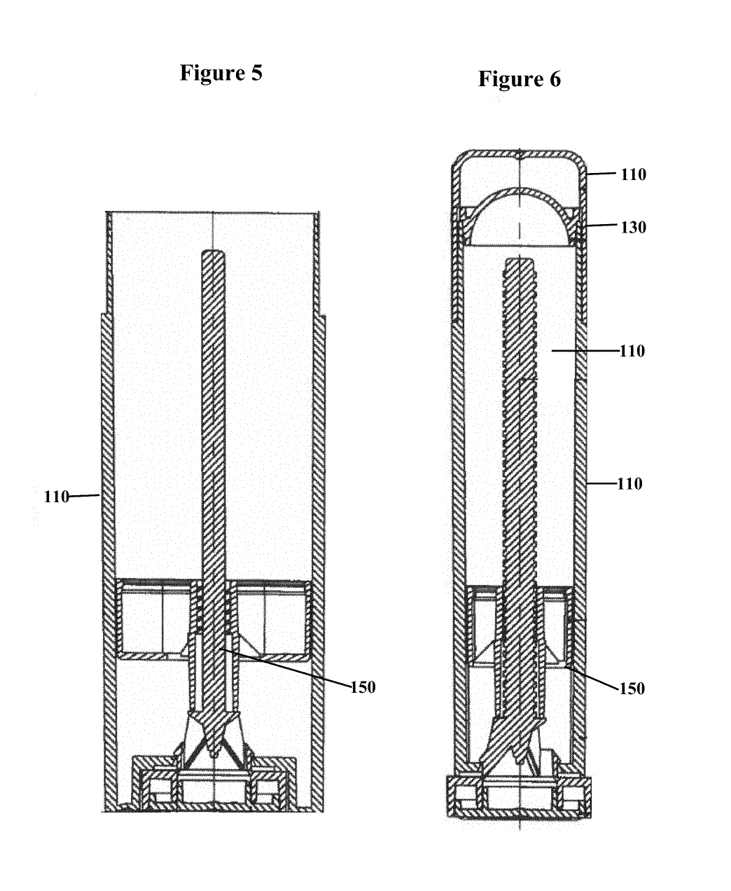

In some embodiments, the cosmetic mask stick product 100, including cosmetic OTC mask stick product 100, will include a soft solid fixed form cosmetic mask formulation 10, including cosmetic OTC mask formulation 10, as described above, that is capable of maintaining its solid shape until applied to the skin; whereupon the composition is paid out in a substantially uniform layer; and a screw dispenser housing containing the soft solid fixed form cosmetic mask formulation, including a cosmetic OTC mask stick formulation. In some instances, the screw dispenser 110 comprises a screw-thread mechanism 150 for selectively advancing the soft solid fixed form cosmetic mask formulation 10 with in and out of the screw dispenser 110. An exemplary screw dispenser 110, as is commonly used for deodorants and other soft solid fixed forms, is shown in FIGS. 1-8. As shown, the screw dispenser employs a screw-thread advancement mechanism 150. The screw-thread is advanced by turning the thumb-wheel 160 at the base of the dispenser. FIG. 7 depicts an outer cap 120, and FIG. 8 depicts an inner cap 130 which can be used to seal the product until use and to help shape the product during manufacturing. Although a screw dispenser is described and depicted herein, any type of dispenser which permits advancement of the mask stick within and out of the dispenser housing may be used. For example, push-up dispensers, push-button dispensers, twist dispensers, or others may be used.

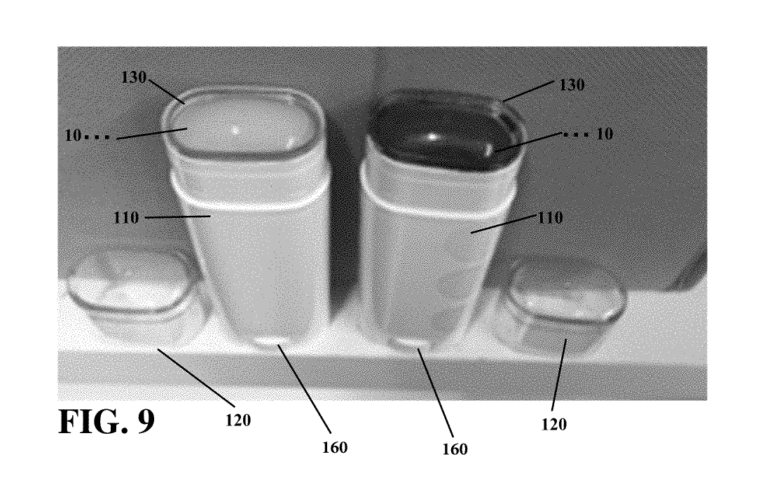

FIG. 9 depicts a photograph of prototype mask stick products. A grapefruit mask stick and a tomato charcoal mask stick product are shown. As depicted throughout the drawings, the mask stick product includes an elongated container having a roughly oval cross-section, an outer cap, and an inner cap. As depicted, the cosmetic mask stick formulation, including cosmetic OTC mask formulation, is housed within the container and can be pushed towards an open end, which is selectively covered by either or both the inner cap and the outer cap. The inner cap serves to help form the mask stick shape and keep the product fresh during pre-purchase storage. The inner cap can be permanently removed by the end user. The outer cap functions as a usable cover to protect the mask stick between uses. As shown, the container is a screw dispenser employing a screw-mechanism which pushes the formulation toward the open end of the container exposing the mask stick formulation for use. As the mask stick formulation is consumed, additional mask stick can be advanced for continued use.

The description above is not intended to be limiting in any way but rather, illustrative of a cosmetic mask, including cosmetic OTC mask, in a soft solid fixed form, often referred to simply as a mask stick, which can be made in a variety of ways, housed in a variety of containers/dispensers, from a variety of ingredients, for a variety of purposes. Only Applicant has recognized that such forms can, and should, be made for cosmetic mask and cosmetic OTC mask application.

We claim:

1. A cosmetic mask stick comprising:
a cosmetic mask formulation comprising:
a skin care and aesthetic portion; and
a structural and functional portion;
wherein said cosmetic mask formulation takes a soft solid fixed form capable of maintaining a solid shape until applied to the skin; whereupon the composition is paid out in a substantially uniform layer; and
a dispenser housing defining an open end and containing the soft solid fixed form cosmetic mask formulation for delivery through the open end.

2. The cosmetic mask stick of claim 1, wherein the dispenser housing is provided with a dispensing mechanism for selectively advancing the soft solid fixed form cosmetic mask formulation within and out of the open end of the dispenser housing.

3. The cosmetic mask stick of claim 1, wherein the dispensing mechanism is a screw-thread or push up mechanism.

4. The cosmetic mask stick of claim 1, wherein the structural and function portion comprises one or more filler, binder, wax, oil, solvent, solubilizer, humectant, hydrogel, surfactant, thickening agent, thinning agent, melting agent, activator, polymer, or other ingredients useful for adding, developing, and maintaining a soft solid fixed form, and any combination thereof.

5. The cosmetic mask stick of claim 1, wherein the cosmetic and aesthetic portion comprises one or more of charcoal, salicylic acid, tomato extract, or a combination thereof.

6. The cosmetic mask stick of claim 1, wherein the cosmetic and aesthetic portion comprises a charcoal, salicylic acid, and a tomato extract.

7. The cosmetic mask stick of claim 1, wherein the cosmetic and aesthetic portion comprises one or more of grapefruit-based ingredient, vitamin C, or a combination thereof.

8. The cosmetic mask stick of claim 1, wherein the cosmetic and aesthetic portion comprises a grapefruit-based ingredient and vitamin C.

9. The cosmetic mask stick of claim 1, further comprising one or more exfoliant.

10. The cosmetic mask stick of claim 1, wherein the skin care and aesthetic portion further comprises one or more moisturizer, exfoliant, purifying agent, charcoal, activated carbon, fruit extract or essence, vegetable extract or essence, essential oil, anti-aging ingredient, enzyme, vitamin, mineral, whitening agent, bronzing agent, spot remover, anti-wrinkling agent, humectant, hyaluronic acid, anti-acne ingredients, or combination thereof.

11. The cosmetic mask stick of claim 1, wherein the cosmetic mask stick formulation, comprises at least about 95% by weight natural ingredients by weight of the formulation, excluding water.

12. A cosmetic stick comprising:
a cosmetic mask composition in a soft solid fixed form.

13. The cosmetic mask stick of claim 1, wherein the cosmetic mask stick is a cosmetic over the counter stick.
